SEQUENCE LISTING (3 sequences)

SEQ ID NO: 1 - DNA, 4 bp, Actinomyces viscosus
atcc

SEQ ID NO: 2 - DNA, 4 bp, Streptococcus mutans
atcc

SEQ ID NO: 3 - DNA, 4 bp, Porphyromonas gingivalis
atcc

(19) United States
(12) Patent Application Publication
(10) Pub. No.: US 2006/0147391 A1
(43) Pub. Date: Jul. 6, 2006

(54) Composition for enhancing oral health

(76) Inventors: Hyung-Jin Kim, Daejeon (KR); Sang-Nyun Kim, Deajeon (KR); Hoo-Deok Kim, Daejeon (KR); Young-Ho Kim, Daejeon (KR); Moon-Moo Kim, Daejeon (KR); Hyoung-Kook Park, Daejeon (KR)

Correspondence Address: INTELLECTUAL PROPERTY GROUP;FREDRIKSON & BYRON, P.A., 200 SOUTH SIXTH STREET, SUITE 4000, MINNEAPOLIS, MN, 55402, US

(21) Appl. No.: 10/514,778

(22) PCT Filed: May 16, 2003
(86) PCT No.: PCT/KR03/00979

§ 371 Date: Aug. 1, 2005

(30) Foreign Application Priority Data
May 16, 2002 (KR) 10-2002-0026958

(51) Int. Cl. A61K 8/97; A61K 36/906

(52) U.S. Cl. 424/48; 424/756; 424/58

(57) ABSTRACT

Disclosed is composition for enhancing oral health, which includes 0.0001˜10 wt % of Xanthorrhizol on the basis of the total weight of the composition, and 0.01˜10 wt % of at least one antibacterial activity enhancer selected form the group consisting of alkyl sodium sulfate, sodium lauroyl sarcocinate and sodium methylcocoyltaurate. The antibacterial activity enhancer contained in the composition more effectively enhances antibacterial activity of Xanthorrhizol against causative bacterial of a decayed tooth or a peridental disease existing in the mouth, so the composition is very helpful for enhancing the oral health, for example the prevention or relief of a decayed tooth or a peridental disease or the restraint of foul breath.

### TECHNICAL FIELD

The present invention relates to a composition for enhancing oral health, and more particularly to a composition for enhancing oral health which well prevents and relives caries and periodontal disease and restrains foul breath by containing Xanthorrhizol and a predetermined antibacterial activity enhancer.

### BACKGROUND ART

Generally, caries and periodontal disease are caused in the following ways. In case of the caries, protein among the saliva in the mouth is absorbed on the surface of dentine and cement to form a film, on which pathogens such as Streptococcus or Actinomyces grow up to create plaque. As time goes, calcium and phosphorous among the saliva are absorbed onto the plaque to form tartar and at the same time the organic acid secreted by those pathogens decalcifies to create enamel and dentine.

On the other hand, the periodontal disease may bring the tooth to be lost due to gingivitis, bleeding, formation of periodontal pocket or breakage of alveolar bone. In aspect of the generation of such periodontal disease, at first some plaque moves toward root apex and at the same time anaerobic gram-negative bacteria such as Porphyromonas and Actinobacillus grow in gingival sulcus, so these bacteria, bacterial constituent components and bacterial products are penetrated into gingival connective tissue through epithelium of the gingival sulcus to form periodontal pocket. As a result of metabolism of the bacterial, there are secreted toxins harmful for cells such as poisonous hydrogen sulfide, ammonia and poisonous amine in the periodontal tissue. In addition, various kinds of Cytokine such as activated oxygen, nitrogen peroxide, Prostaglandins, Leukotriens, Histamine, Interleuckins and Tumour necrosis factor a secreted out of cells due to various actions of stimulated humoral and cellular immune system generated by stimulating biological immune system cause gingival inflammation, and enzyme such as Collagenase secreted from bacteria and leukocytes dissolve Collagen which is a stroma of the periodontal tissue, thereby causing involution of the gum, which brings periodontal disease when left as it is.

In order to prevent and cure the caries and periodontal disease, there are generally used a method using antibiotics such as Spiramycin, Vancomycin and Chlorhexidine; and a method for restraining bacteria arousing caries and periodontal disease by using organic or inorganic fluorine. The method using antibiotics is effective in preventing the caries, but it may cause generation of culture having tolerance to antibiotics depending on the kind of used antibiotic and disadvantageously give rise to ill effects such as diarrhea and emesis.

Thus, there have been continued the endeavor for separating caries-tolerant and periodontal disease-tolerant substances having no ill effect from natural resources such as medicinal plants or herbs. Several kinds of Tannin compounds having inhibitory activity to Glucosyl transferase have ever been extracted from Cacao bean and Persimmon leaves. However, these compounds have many limitations in the substantial industrial application since it is reported that these Tannin compounds nonspecifically deteriorate most of the enzyme actions in addition to Glucosyl transferase.

Recently, there is reported an experimental result that Xanthorrhizol contained in extracts extracted from Curcuma xanthorrhiza Roxb. shows excellent antibacterial activity against oral microorganisms (see Korean Laid-open Patent Publication Nos. 2000-0000256, 2000-0000342, 2000-0006939 and 2000-0073295, and WO 00/67711). However, this is just a result in vitro, and this composition for enhancing oral health cannot stay in the mouth for a long time since a user spits out it after washing, so it is difficult for the composition to sufficiently prevent and relieve caries and periodontal disease. Thus, there still need researches for more improving the antibacterial activity of Xanthorrhizol.

### DISCLOSURE OF INVENTION

The present invention is designed to solve the problems of the prior art, and therefore an object of the invention is to provide a composition for enhancing oral health which is capable of better preventing and reliving caries and periodontal disease and restraining foul breath more effectively by containing a predetermined surface active agent as an antibacterial activity enhancer for improving antibacterial activity of Xanthorrhizol.

In order to accomplish the above object, the present invention provides a composition for enhancing oral health, which includes 0.0001˜10 wt % of Xanthorrhizol on the basis of the total weight of the composition; and 0.01˜10 wt % of at least one antibacterial enhancer selected from the group consisting of alkyl sodium sulfate, sodium lauroyl sarcocinate and sodium methylcocoyltaurate.

In another aspect of the present invention, there is also provide a composition for enhancing oral health, which includes 0.001˜20 wt % of Curcuma Xanthorrhiza Roxb. extracts on the basis of the total weight of the composition; and 0.01˜10 wt % of at least one antibacterial enhancer selected from the group consisting of alkyl sodium sulfate, sodium lauroyl sarcocinate and sodium methylcocoyltaurate.

Preferably, the composition is formulated into a dosage form selected from the group consisting of gargling water, toothpaste and edible breath film.

### BEST MODES FOR CARRYING OUT THE INVENTION

Hereinafter, a composition for enhancing oral health according to the present invention will be described in detail.

Curcuma Xanthorrhiza Roxb. belongs to the ginger family, and it is known to have various physiological activities such as serum cholesterol decreasing action, antitumor action, anti-inflammation effects and wound curing effects. Curcuma Xanthorrhiza Roxb. mainly contains α-curcumene, β-curcumene, arturmenone, xanthorrhizol, germacrone, β-sesquiphellandrene, curzerenone, α-turmerone, β-turmerone and so on. Among them, Xanthorrhizol is particularly reported to have excellent antibacterial activity to intraoral bacteria such as Streptococcus mutans, Streptococcus sobrinus and Porphyromonas gingivalis.

To obtain Curcuma Xanthorrhiza Roxb. extracts, various ways are already known in the art. Supercritical fluid extracting, microwave extracting and supersonic wave extracting may be used in addition to the general organic solvent extracting. In addition, the traditional expression or hot water extracting may be used to obtain oil constituents (see WO 88/05304 and WO 00/67711). To explain the organic solvent extracting as an example, Curcuma Xanthorrhiza Roxb. extracts is manufactured using the following processes: a) drying and grinding purified Curcuma Xanthorrhiza Roxb., b) conducting extraction by putting an extracting solvent into the pulverized Curcuma Xanthorrhiza Roxb., c) filtering the extracted stock solution, d) condensing the filtered extracted stock solution, and e) freeze-drying the condensed stock solution into dried powder. In particular, considering that activating components are useful among various solvents, it is preferably to conduct the extracting process by use of n-hexane, ethanol or the like. At this time, it is also preferred to conduct the extracting process so that 10˜99 wt % of Xanthorrhizol which is an effective constituent among Curcuma Xanthorrhiza Roxb. extracts is contained on the basis of the total solid matters. In addition, it is also possible to use high purity Xanthorrhizol purified using the way disclosed in WO 00-67711.

In the composition for enhancing oral health according to the present invention, it is preferred that 0.0001˜10 wt % of Xanthorrhizol, which is an antibacterial constituent to causative bacteria of caries and gingival disease existing in the mouth, is contained on the basis of the total weight of the composition, in the consideration of antibacterial activity and cost. In addition, it is also preferred that 0.001˜20 wt % of Curcuma Xanthorrhiza Roxb. containing Xanthorrhizol is contained on the basis of the total weight of the composition.

The composition for enhancing oral health according to the present invention also contains at least one of alkyl sodium sulfate such as lauroyl sodium sulfate, sodium lauroyl sarcocinate and sodium methylcocoyltaurate, as an antibacterial activity enhancer. In case of using alkyl sodium sulfate as an antibacterial activity enhancer, the number of carbons in the alkyl group is preferably 8˜22.

The surface active agent used as a foaming agent plays roles of assisting the washing action of an abrasive, penetrating medical active agents into the regions where a toothbrush hardly reaches, and improving the brushing feeling of the teeth by generating foams, but cannot increase pharmacological action of Xanthorrhizol. However, alkyl sodium sulfate, sodium lauroyl sarcocinate and sodium methylcocoyltaurate contained in the oral health enhancing composition of the present invention may increase antibacterial activity of Xanthorrhizol, thereby improving the oral health enhancing effects such as prevention and relief of caries and periodontal disease and restraint of foul breath. Preferably, there is contained 0.01˜10 wt % of such an antibacterial activity enhancer on the basis of the total weight f the composition.

In the composition for enhancing oral health according to the present invention, components other than Curcuma Xanthorrhiza Roxb. extracts and the antibacterial activity enhancer are suitably mixed in a common way depending on their kinds and usages. Hereinafter, abrasive, fluorine compounds, wetting agent and binding agent, which may be added to toothpaste composition as an example of the oral health enhancing composition of the present invention, are described.

The abrasive may include constituents mixing at least one of dicalcium hydrogenphosphate, precipitated silica, sodium hydrogencarbonate, calcium carbonate, alumina hydrate, insoluble sodium metaphosphate, rigid calcium carbonate, sodium pyrophosphate and silica gel, and 1˜90 wt % of this abrasive may be used. In addition, as for the fluorine compound which is used as a medicinal effective agent for reinforcing tooth tissue by promoting recalcification of the tooth, at least one of sodium fluoride, monosodium phosphate fluoride (or, sodium phosphate fluoride, monobasic) and stannous fluoride may be used, and its suitable amount is 0.01˜2.0 wt %.

The wetting agent is used for keeping the state of toothpaste composition and preventing the toothpaste composition from drying. The wetting agent contains at least one selected from the group consisting of polyethylene glycol, propylene glycol, sorbitol and glycerin, and its content may be 20˜60 wt %. In addition, the binding agent is used for keeping the formation of toothpaste and securing its stability by binding liquid components and solid components of the toothpaste. Mainly sodium carrageenate, calcium salt, sodium carboxymethylcellulose, or natural or synthetic polymer substances such as xanthan gum or acacia gum may be used for the binding agent, and 0.1˜5 wt % of binding agent is preferably used.

Aromatics or sweetening agents are usually used for somewhat unpleasant and bitter taste. As the aromatics, peppermint or spearmint oil which is natural pigment is mainly used, and its content may be 0.1˜1 wt %. As the sweetening agent, synthetic or natural non-fermentable sugar is mainly used, and representatively 0.05˜1 wt % of sodium saccharine, aspartame, lactose or stevioside may be used.

As a buffering agent for adjusting pH of the toothpaste composition, there are monosodium phosphate (or, sodium phosphate, monobasic), disodium phosphate (or, sodium phosphate, dibasic), trisodium phosphate (or, sodium phosphate, tribasic), citric acid, sodium citric acid, tartaric acid and so on. In addition, in order to prevent contamination of microorganisms which may occur during manufacture or usage of the toothpaste composition, there may be used an antiseptic such as methyl parahydroxybenzoate, propyl parahydroxybenzoate or sodium benzoate.

The oral health enhancing composition according to the present invention is made by mixing the antibacterial activity enhancer and Xanthorrhizol showing excellent anti bacterial activity or Curcuma Xanthorrhiza Roxb. extracts containing such Xanthorrhizol with a suitable amount of carrier component commonly used in its product.

Now, the present invention is described in more detail with examples of toothpaste, gargling water and edible breath film which are universal dosage forms of the composition for enhancing oral health. However, the technical scope of the present invention is not limited to those examples, but the oral health enhancing composition of the present invention may be applied to gum, chocolate, candy, ointment, mouth spray, mouth rinses, tablet, hygienic tablet and skin patch.

### EMBODIMENTS 1 TO 8 & COMPARATIVE EXAMPLES 1 TO 4

Mouth rinse containing Curcuma Xanthorrhiza Roxb. extracts or Xanthorrhizol is made according to components and contents stated in the following Tables 1 and 2 by use of a common mouth rinse making method.

Curcuma Xanthorrhiza Roxb. extracts or Xanthorrhizol used in the embodiments 1 to 8 are made in the following way. Curcuma Xanthorrhiza Roxb. herb is dried in the shade and finely cut into powder, and then 500 ml of 95% ethanol is applied to 50 g of the powder, which is then deposited and extracted for three days. The extract is then filtered using Whatman No. 1 and then vacuum-concentrated (30 wt % of Xanthorrhizol is contained). In addition, Xanthorrhizol is separated and purified according to the way disclosed in WO 00/67711. On the other hand, alkyl sodium bisulfite has the carbon number of 16.

TABLE 1
(100 g based, %)

| | | | | | | | Experimental | |
|---|---|---|---|---|---|---|---|---|
| | | | Embodiments | | | | examples | |
| Raw material | | Name of raw material | 1 | 2 | 3 | 4 | 1 | 2 |
| | | Curcuma Xanthorrhiza Roxb. | 0.03 | 0.03 | 0.03 | 0.03 | 0.03 | 0.03 |
| | | extracts | | | | | | |
| Antibacterial | Surface | sodium lauryl sulfate | 0.4 | 0.4 | — | — | — | — |
| activity | active | alkyl sodium sulfate | — | — | 0.4 | — | — | — |
| enhancer | agent | (decyl sodium sulfate) | | | | | | |
| | | sodium lauroyl sarcocinate | — | — | — | 0.3 | — | — |
| | | sodium methylcocoyltaurate | — | 0.2 | 0.2 | 0.1 | — | — |
| | nonionic | polyoxyethylene copolymer | — | — | — | — | 1.0 | 0.5 |
| | | hydrogenated castor oil | — | — | — | — | — | 0.5 |

| | | | | | | | |
|---|---|---|---|---|---|---|---|
| Base | glycerin | 5.0 | 5.0 | 5.0 | 5.0 | 5.0 | 5.0 |
| PH adjuster | citric acid | 0.1 | 0.1 | 0.1 | 0.1 | 0.1 | 0.1 |
| | sodium citric acid | 0.2 | 0.2 | 0.2 | 0.2 | 0.2 | 0.2 |
| Favorings | sodium saccharine | 0.01 | 0.01 | 0.01 | 0.01 | 0.01 | 0.01 |
| | perfume | 0.15 | 0.15 | 0.15 | 0.15 | 0.15 | 0.15 |
| Solvent | ethanol | 12.0 | 12.0 | 12.0 | 12.0 | 12.0 | 12.0 |
| | Purified water | to 100 | to 100 | to 100 | to 100 | to 100 | to 100 |

TABLE 2
(100 g based, %)

| | | | | | | | Experimental | |
|---|---|---|---|---|---|---|---|---|
| | | | Embodiments | | | | examples | |
| Raw material | | Name of raw material | 5 | 6 | 7 | 8 | 3 | 4 |
| | | Curcuma Xanthorrhiza Roxb. | 0.03 | 0.03 | 0.03 | 0.03 | 0.03 | 0.03 |
| | | extracts | | | | | | |
| Antibacterial | surface | sodium lauryl sulfate | 0.4 | 0.4 | — | — | — | — |
| activity | active | alkyl sodium sulfate | — | — | 0.4 | — | — | — |
| enhancer | agent | (decyl sodium sulfate) | | | | | | |
| | | sodium lauroyl sarcocinate | — | — | — | 0.3 | — | — |
| | | sodium methylcocoyltaurate | — | 0.2 | 0.2 | 0.1 | — | — |
| | nonionic | polyoxyethylene copolymer | — | — | — | — | 1.0 | 0.5 |
| | | hydrogenated castor oil | — | — | — | — | — | 0.5 |

| | | | | | | | |
|---|---|---|---|---|---|---|---|
| Base | glycerin | 5.0 | 5.0 | 5.0 | 5.0 | 5.0 | 5.0 |
| PH adjuster | citric acid | 0.1 | 0.1 | 0.1 | 0.1 | 0.1 | 0.1 |
| | sodium citric acid | 0.2 | 0.2 | 0.2 | 0.2 | 0.2 | 0.2 |
| Favorings | sodium saccharine | 0.01 | 0.01 | 0.01 | 0.01 | 0.01 | 0.01 |
| | perfume | 0.15 | 0.15 | 0.15 | 0.15 | 0.15 | 0.15 |
| | ethanol | 12.0 | 12.0 | 12.0 | 12.0 | 12.0 | 12.0 |
| Solvent | Purified water | to 100 | to 100 | to 100 | to 100 | to 100 | to 100 |

Experiments for Testing Antibacterial Effects

In order to verify the antibacterial effects to oral microorganisms of the mouth rinse compositions according to the embodiments 1 to 8 and the comparative examples 1 to 4, the following experiment is conducted.

In the experiment, three kinds of cultures such as Actinomyces viscosus (ATCC 15987), Streptococcus mutans (ATCC 10449) and Porphyromonas gingivalis (ATCC 381) are used.

Among these cultures, Actinomyces viscosus (ATCC 15987) is cultivated in YM culture medium (glucose 10 g/L, yeast extract 3 g/L, malt extract 3 g/L, bactopeptone 5 g/L), Streptococcus mutans (ATCC 10449) is cultivated in TH culture medium (Todd Hewitt broth 30 g/L, sucrose 50 g/L), and Porphyromonas gingivalis (ATCC 381) is cultivated in Thioglycollate culture medium. As for the cultivating temperature, Actinomyces viscosus (ATCC 15987) is cultivated in an anaerobic culture medium at 26° C., Streptococcus mutans (ATCC 10449) is cultivated at 37° C., and Porphyromonas gingivalis (ATCC 381) is cultivated in an anaerobic culture medium at 37° C.

The antibacterial activity test is conducted after subculturing these cultures and then preculturing them for 24 hours three times. At this time, 0.1 ml of the medicinal effective agent according to each of the above embodiments and comparative examples is added to 9.9 ml of a suitable culture medium, and then 1×10⁶ cultures are inoculated. After that, it is cultivated for 18 hours, and then the number is measured in the colony count by using the agar dilution method, as shown in Tables 3 and 4.

TABLE 3

| | | Actinomyces | Streptococcus | Porphyromonas |
|---|---|---|---|---|
| | | viscosus | mutans | gingivalis |
| Embodiments | 1 | 0* | 0 | 0 |
| | 2 | 0 | 0 | 0 |
| | 3 | 0 | 0 | 0 |
| | 4 | 0 | 0 | 0 |
| Comparative | 1 | 6 × 10⁷ | 3 × 10⁹ | 2 × 10⁶ |
| examples | 2 | 4 × 10⁸ | 2 × 10⁹ | 2 × 10⁷ |

(0* is less than 10 bacteria.)

TABLE 4

| | | Actinomyces | Streptococcus | Porphyromonas |
|---|---|---|---|---|
| | | viscosus | mutans | gingivalis |
| Embodiments | 5 | 0* | 0 | 0 |
| | 6 | 0 | 0 | 0 |
| | 7 | 0 | 0 | 0 |
| | 8 | 0 | 0 | 0 |
| Comparative | 3 | 7 × 10⁷ | 3 × 10⁹ | 3 × 10⁶ |
| examples | 4 | 4 × 10⁸ | 3 × 10⁹ | 2 × 10⁷ |

(0* is less than 10 bacteria.)

Referring to Tables 3 and 4, it will be understood that the mouse rinses of the embodiments 1 to 8 containing Xanthorrhizol or Curcuma Xanthorrhiza Roxb. and the antibacterial activity enhancer of the present invention show very excellent antibacterial effects against the three kinds of used cultures.

On the other hand, the mouth rinses of the comparative examples 1 to 4 not containing the antibacterial activity enhancer of the present invention show not so good antibacterial effects.

### EMBODIMENTS 9 TO 22 & COMPARATIVE EXAMPLES 5 AND 6

Toothpaste compositions containing Curcuma Xanthorrhiza Roxb. extracts or Xanthorrhizol as an antibacterial component is made according to components and contents stated in the following Tables 5 and 6 by use of a common toothpaste composition making method.

TABLE 5
(100 g based, %)

| | | | | | | | | | Comparative |
|---|---|---|---|---|---|---|---|---|---|
| | | Embodiments | | | | | | | examples |
| Raw material | Name of raw material | 9 | 10 | 11 | 12 | 13 | 14 | 15 | 5 |
| | Curcuma Xanthorrhiza Roxb. | 0.4 | 0.4 | 0.4 | 0.4 | 0.4 | 0.4 | 0.4 | 0.4 |
| | extracts | | | | | | | | |
| Antibacterial | sodium lauryl sulfate | 2 | — | — | — | 1 | — | — | — |
| activity enhancer | alkyl sodium sulfate | — | 2 | — | — | — | 1 | — | — |
| | (decyl sodium sulfate) | | | | | | | | |
| | sodium lauroyl sarcocinate | — | — | 2 | — | — | — | 1 | — |
| | sodium methylcocoyltaurate | — | — | — | 2 | 1 | 1 | 1 | — |
| Abrasive | calcium carbonate | 40.0 | 40.0 | 40.0 | 40.0 | 40.0 | 40.0 | 40.0 | 40.0 |
| Wetting agent | sorbitol liquid | 25.0 | 25.0 | 25.0 | 25.0 | 25.0 | 25.0 | 25.0 | 25.0 |
| Foaming agent | polyoxyethylene copolymer | 2.0 | 2.0 | 2.0 | 2.0 | 2.0 | 2.0 | 2.0 | 2.0 |
| Binding agent | carboxymethylcellulose | 1.0 | 1.0 | 1.0 | 1.0 | 1.0 | 1.0 | 1.0 | 1.0 |
| Favoring | sodium saccharine | 0.2 | 0.2 | 0.2 | 0.2 | 0.2 | 0.2 | 0.2 | 0.2 |
| Antiseptic | ester parahydroxybenzoate | 0.1 | 0.1 | 0.1 | 0.1 | 0.1 | 0.1 | 0.1 | 0.1 |
| Aromatics | synthetic aromatics | 1.0 | 1.0 | 1.0 | 1.0 | 1.0 | 1.0 | 1.0 | 1.0 |
| Buffering liquid | monosodium phosphate | 0.1 | 0.1 | 0.1 | 0.1 | 0.1 | 0.1 | 0.1 | 0.1 |
| | trisodium phosphate | 0.05 | 0.05 | 0.05 | 0.05 | 0.05 | 0.05 | 0.05 | 0.05 |
| | Purified water | to 100 | to 100 | to 100 | to 100 | to 100 | to 100 | to 100 | to 100 |

TABLE 6
(100 g based, %)

| | | | | | | | | | Comparative |
|---|---|---|---|---|---|---|---|---|---|
| | | Embodiments | | | | | | | examples |
| Raw material | Name of raw material | 16 | 17 | 18 | 19 | 20 | 21 | 22 | 6 |
| | Curcuma Xanthorrhiza Roxb. | 0.3 | 0.3 | 0.3 | 0.3 | 0.3 | 0.3 | 0.3 | 0.3 |
| | extracts | | | | | | | | |
| Antibacterial | sodium lauryl sulfate | 2 | — | — | — | 1 | — | — | — |
| activity enhancer | alkyl sodium sulfate | — | 2 | — | — | — | 1 | — | — |
| | (decyl sodium sulfate) | | | | | | | | |
| | sodium lauroyl sarcocinate | — | — | 2 | — | — | — | 1 | — |
| | sodium methylcocoyltaurate | — | — | — | 2 | 1 | 1 | 1 | — |
| Abrasive | calcium carbonate | 40.0 | 40.0 | 40.0 | 40.0 | 40.0 | 40.0 | 40.0 | 40.0 |
| Wetting agent | sorbitol liquid | 25.0 | 25.0 | 25.0 | 25.0 | 25.0 | 25.0 | 25.0 | 25.0 |
| Foaming agent | polyoxyethylene copolymer | 2.0 | 2.0 | 2.0 | 2.0 | 2.0 | 2.0 | 2.0 | 2.0 |
| Binding agent | carboxymethylcellulose | 1.0 | 1.0 | 1.0 | 1.0 | 1.0 | 1.0 | 1.0 | 1.0 |
| Favoring | sodium saccharine | 0.2 | 0.2 | 0.2 | 0.2 | 0.2 | 0.2 | 0.2 | 0.2 |
| Antiseptic | ester parahydroxybenzoate | 0.1 | 0.1 | 0.1 | 0.1 | 0.1 | 0.1 | 0.1 | 0.1 |
| Aromatics | synthetic aromatics | 1.0 | 1.0 | 1.0 | 1.0 | 1.0 | 1.0 | 1.0 | 1.0 |
| Buffering liquid | monosodium phosphate | 0.1 | 0.1 | 0.1 | 0.1 | 0.1 | 0.1 | 0.1 | 0.1 |
| | trisodium phosphate | 0.05 | 0.05 | 0.05 | 0.05 | 0.05 | 0.05 | 0.05 | 0.05 |
| | Purified water | to 100 | to 100 | to 100 | to 100 | to 100 | to 100 | to 100 | to 100 |

Experiments for Testing Antibacterial Effects

In order to verify the antibacterial effects to oral microorganisms of the toothpaste compositions according to the embodiments 9 to 22 and the comparative examples 5 and 6, the following experiment is conducted in the same way as the former experiment for the mouse rinses.

In the experiment, three kinds of cultures such as Actinomyces viscosus (ATCC 15987), Streptococcus mutans (ATCC 10449) and Porphyromonas gingivalis (ATCC 381) are used.

The antibacterial activity test is conducted after subculturing these cultures and then preculturing them for 24 hours three times. At this time, 0.1 ml of the medicinal effective agent according to each of the above embodiments and comparative examples is added to 9.9 ml of a suitable culture medium, and then 1×10⁶ cultures are inoculated. After that, it is cultivated for 18 hours, and then the number is measured in the colony count by using the agar dilution method, as shown in Tables 7 and 8.

TABLE 7

| | | Actinomyces | Streptococcus | Porphyromonas |
|---|---|---|---|---|
| | | viscosus | mutans | gingivalis |
| Embodiments | 9 | 80 | 70 | 85 |
| | 10 | 70 | 50 | 40 |
| | 11 | 75 | 70 | 50 |
| | 12 | 90 | 80 | 80 |
| | 13 | 50 | 60 | 40 |
| | 14 | 60 | 55 | 30 |
| | 15 | 55 | 40 | 45 |
| Comparative | 5 | 6 × 10⁷ | 3 × 10⁹ | 3 × 10⁶ |
| examples | | | | |

TABLE 8

| | | Actinomyces | Streptococcus | Porphyromonas |
|---|---|---|---|---|
| | | viscosus | mutans | gingivalis |
| Embodiments | 16 | 0* | 0 | 0 |
| | 17 | 0 | 0 | 0 |
| | 18 | 0 | 0 | 0 |
| | 19 | 10 | 10 | 10 |
| | 20 | 0 | 0 | 0 |
| | 21 | 0 | 0 | 0 |
| | 22 | 0 | 0 | 0 |
| Comparative | 6 | 6 × 10⁷ | 3 × 10⁹ | 2 × 10⁶ |
| examples | | | | |

(0* is less than 10 bacteria.)

Referring to Tables 7 and 8, it will be understood that the toothpaste of the embodiments 9 to 22 containing Xanthorrhizol or Curcuma Xanthorrhiza Roxb. together with the antibacterial activity enhancer of the present invention show very excellent antibacterial effects against the three kinds of used cultures.

On the other hand, the toothpaste of the comparative examples 5 and 6 not containing the antibacterial activity enhancer of the present invention show not so good antibacterial effects.

### EMBODIMENTS 23 TO 38 & COMPARATIVE EXAMPLES 7 TO 10

Edible breath films containing Curcuma Xanthorrhiza Roxb. extracts or Xanthorrhizol as an antibacterial component is made according to components and contents stated in the following Tables 7 and 8 by use of a common edible breath film making method.

TABLE 9
(100 g based, %)

| | | | | | | | | | Comparative | |
|---|---|---|---|---|---|---|---|---|---|---|
| | Embodiments | | | | | | | | examples | |
| Name of raw material | 23 | 24 | 25 | 26 | 27 | 28 | 29 | 30 | 7 | 8 |
| Curcuma Xanthorrhiza Roxb. | 0.3 | 0.3 | 0.3 | 0.3 | 0.3 | 0.3 | 0.3 | 0.3 | 0.3 | 0.3 |
| extracts | | | | | | | | | | |
| sodium lauryl sulfate | 0.1 | 0.1 | — | — | 0.1 | 0.1 | — | — | — | — |
| alkyl sodium sulfate | — | — | 0.1 | — | — | — | 0.1 | — | — | — |
| (decyl sodium sulfate) | | | | | | | | | | |
| sodium lauroyl sarcocinate | — | — | — | 0.1 | — | — | — | 0.1 | — | — |
| sodium methylcocoyltaurate | — | 0.1 | 0.1 | 0.1 | — | 0.1 | 0.1 | 0.1 | — | — |
| pullulan | 20 | 20 | 20 | 20 | — | — | — | — | 20 | — |
| sodium alginate | 20 | 20 | 20 | 20 | 20 | 20 | 20 | 20 | — | 20 |
| Twin 80 | 0.1 | 0.1 | 0.1 | 0.1 | 0.1 | 0.1 | 0.1 | 0.1 | 0.1 | 0.1 |
| carrageenan | 0.1 | 0.1 | 0.1 | 0.1 | 0.1 | 0.1 | 0.1 | 0.1 | 0.1 | 0.1 |
| Low cost bean | 0.1 | 0.1 | 0.1 | 0.1 | 0.1 | 0.1 | 0.1 | 0.1 | 0.1 | 0.1 |
| xanthan gum | 1 | 1 | 1 | 1 | 1 | 1 | 1 | 1 | 1 | 1 |
| Perfume | 1 | 1 | 1 | 1 | 1 | 1 | 1 | 1 | 1 | 1 |
| Purified water | to 100 | to 100 | to 100 | to 100 | to 100 | to 100 | to 100 | to 100 | to 100 | to 100 |

TABLE 10
(100 g based, %)

| | | | | | | | | | Comparative | |
|---|---|---|---|---|---|---|---|---|---|---|
| | Embodiments | | | | | | | | examples | |
| Name of raw material | 31 | 32 | 33 | 34 | 35 | 36 | 37 | 38 | 9 | 10 |
| Curcuma Xanthorrhiza Roxb. | 0.2 | 0.2 | 0.2 | 0.2 | 0.2 | 0.2 | 0.2 | 0.2 | 0.2 | 0.2 |
| extracts | | | | | | | | | | |
| sodium lauryl sulfate | 0.1 | 0.1 | — | — | 0.1 | 0.1 | — | — | — | — |
| alkyl sodium sulfate | — | — | 0.1 | — | — | — | 0.1 | — | — | — |
| (decyl sodium sulfate) | | | | | | | | | | |
| sodium lauroyl sarcocinate | — | — | — | 0.1 | — | — | — | 0.1 | — | — |
| sodium methylcocoyltaurate | — | 0.1 | 0.1 | 0.1 | — | 0.1 | 0.1 | 0.1 | — | — |
| pullulan | 20 | 20 | 20 | 20 | — | — | — | — | 20 | — |
| sodium alginate | 20 | 20 | 20 | 20 | 20 | 20 | 20 | 20 | — | 20 |
| Twin 80 | 0.1 | 0.1 | 0.1 | 0.1 | 0.1 | 0.1 | 0.1 | 0.1 | 0.1 | 0.1 |
| carrageenan | 0.1 | 0.1 | 0.1 | 0.1 | 0.1 | 0.1 | 0.1 | 0.1 | 0.1 | 0.1 |
| Low cost bean | 0.1 | 0.1 | 0.1 | 0.1 | 0.1 | 0.1 | 0.1 | 0.1 | 0.1 | 0.1 |
| xanthan gum | 1 | 1 | 1 | 1 | 1 | 1 | 1 | 1 | 1 | 1 |
| Perfume | 1 | 1 | 1 | 1 | 1 | 1 | 1 | 1 | 1 | 1 |
| Purified water | to 100 | to 100 | to 100 | to 100 | to 100 | to 100 | to 100 | to 100 | to 100 | to 100 |

Experiments for Testing Antibacterial Effects

In order to verify the antibacterial effects to oral microorganisms of the edible breath film compositions according to the embodiments 23 to 38 and the comparative examples 7 to 10, the following experiment is conducted in the same way as the former experiment for the mouse rinses or the toothpaste compositions.

In the experiment, three kinds of cultures such as Actinomyces viscosus (ATCC 15987), Streptococcus mutans (ATCC 10449) and Porphyromonas gingivalis (ATCC 381) are used.

The antibacterial activity test is conducted after subculturing these cultures and then preculturing them for 24 hours three times. At this time, 0.1 ml of the medicinal effective agent according to each of the above embodiments and comparative examples is added to 9.9 ml of a suitable culture medium, and then 1×10⁶ cultures are inoculated. After that, it is cultivated for 18 hours, and then the number is measured in the colony count by using the agar dilution method, as shown in Tables 11 and 12.

TABLE 11

| | | Actinomyces | Streptococcus | Porphyromonas |
|---|---|---|---|---|
| | | viscosus | mutans | gingivalis |
| Embodiments | 23 | 120 | 100 | 95 |
| | 24 | 70 | 40 | 50 |
| | 25 | 65 | 50 | 45 |
| | 26 | 80 | 55 | 50 |
| | 27 | 110 | 100 | 90 |
| | 28 | 70 | 70 | 60 |
| | 29 | 70 | 60 | 40 |
| | 30 | 60 | 80 | 20 |
| Comparative | 7 | 5 × 10⁷ | 2 × 10⁹ | 3 × 10⁶ |
| examples | 8 | 6 × 10⁷ | 3 × 10⁹ | 3 × 10⁶ |

TABLE 12

| | | Actinomyces | Streptococcus | Porphyromonas |
|---|---|---|---|---|
| | | viscosus | mutans | gingivalis |
| Embodiments | 31 | 10 | 10 | 20 |
| | 32 | 0* | 0 | 0 |
| | 33 | 0 | 0 | 0 |
| | 34 | 0 | 0 | 0 |
| | 35 | 20 | 10 | 30 |
| | 36 | 0 | 0 | 0 |
| | 37 | 0 | 0 | 0 |
| | 38 | 0 | 0 | 0 |
| Comparative | 9 | 6 × 10⁷ | 2 × 10⁹ | 3 × 10⁶ |
| examples | 10 | 5 × 10⁷ | 3 × 10⁹ | 4 × 10⁶ |

(0* is less than 10 bacteria.)

Referring to Tables 11 and 12, it will be understood that the edible breath films of the embodiments 23 to 38 containing Xanthorrhizol or Curcuma Xanthorrhiza Roxb. together with the antibacterial activity enhancer of the present invention show very excellent antibacterial effects against the three kinds of used cultures.

On the other hand, the edible breath films of the comparative examples 7 to 10 not containing the antibacterial activity enhancer of the present invention show not so good antibacterial effects.

### INDUSTRIAL APPLICABILITY

As described above, the antibacterial activity enhancer contained in the composition for enhancing oral health according to the present invention more effectively gives antibacterial activity to the causative bacteria of caries and gingival diseases existing in the mouth, so the composition of the present invention may improve the oral health enhancing effects, for example the prevention or relief of caries or periodontal disease or the restraint of foul breath.

The present invention has been described in detail. However, it should be understood that the detailed description and specific examples, while indicating preferred embodiments of the invention, are given by way of illustration only, since various changes and modifications within the spirit and scope of the invention will become apparent to those skilled in the art from this detailed description.

What is claimed is:

1. A composition for enhancing oral health comprising:
0.0001˜10 wt % of Xanthorrhizol on the basis of the total weight of the composition; and
0.01˜10 wt % of antibacterial enhancer including sodium lauroyl sarcocinate and sodium methylcocoyltaurate.

2. A composition for enhancing oral health comprising:
0.001˜20 wt % of Curcuma Xanthorrhiza Roxb. extracts on the basis of the total weight of the composition; and
0.01˜10 wt % of antibacterial enhancer including sodium lauroyl sarcocinate and sodium methylcocoyltaurate.

3. (canceled)

4. A composition for enhancing oral health according to claim 1 or 2,
wherein the composition is formulated into a dosage form selected from the group consisting of gargling water, toothpaste and edible breath film.
